(19) United States
(12) Patent Application Publication
(10) Pub. No.: US 2008/0078415 A1
(43) Pub. Date: Apr. 3, 2008

(54) Dol-Fins: "Swimming with Dol-Fins"

(76) Inventor: Kathleen Mishler, Memphis, TN (US)

Correspondence Address: Kathleen Mishler, Apartment 808, 6 South McLean Boulevard, Memphis, TN, 38104, US

(21) Appl. No.: 11/522,541

(22) Filed: Sep. 18, 2006

(51) Int. Cl. A61F 9/00

(52) U.S. Cl. 128/858

(57) ABSTRACT

This nose-plug is unique in design while at the same time effectively prevents water from entering the nasal cavities of the swimmer. It is made of lightweight wire with a soft covering, which makes it comfortable to wear, and flexible enough to fit almost any size of person. The nose-plug is inconspicuous due to both its small size and by the fact that the covering can be made in a wide variety of skin tones. The nose-plug could be used for both casual and lap swimming, and is capable of staying on the nose even after diving in the water. This device is new to the realm of athletics because it keeps water out of the sinus cavities more effectively than the previous devices.

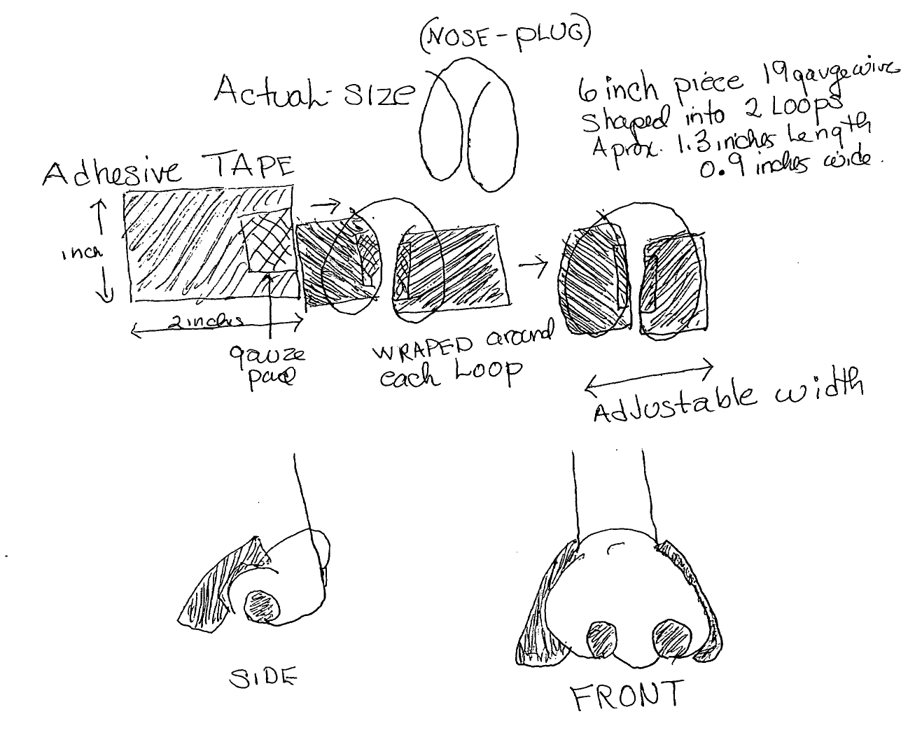

This is a substitute specification for application Ser. No. 11/522,541, which was originally filed on Sep. 18, 2006. This specification contains no new matter from what was contained in the original specification.

The device is a nose-plug designed especially for swimmers that prevents water from entering the nose, which prevents the sinus from becoming irritated. This nose-plug, designed by a serious swimmer, is an improvement upon existing models because it neither pinches the nostrils, nor fits on top of the nostrils, but rather fits behind the nostrils in order to close the openings to the sinus cavities. This nose-plug is durable, long lasting, lightweight, and fits all sizes. In addition, the cloth tape that is used to make the nose-plug can be printed in various flesh tones, therefore making it unobtrusive for people of all skin color.

### BACKGROUND OF THE INVENTION

I have been a swimmer for over 50 years. I have developed this nose-plug out of necessity because the ones currently on the market are inadequate. These devices are made from hard plastic that is uncomfortable, pinches the nostrils, and do not keep the water from entering the nose and nasal cavities. The proposed nose-plug can be used during swimming and diving in deep water, can be comfortably worn for long periods of time, and is not very conspicuous.

### BRIEF SUMMARY OF THE INVENTION

The proposed nose-plug has an advantage over the current models on the market because it is adaptable, long lasting, and does a better job of preventing water from entering the nasal cavities. By using my device, swimmers can help prevent the sinus irritation and infection that is caused when chemicals used in the swimming pool enter the nasal cavities.

### DETAILED DESCRIPTION OF THE INVENTION

The nose-plug is created by taking a 6 inch piece of 19 gauge wire and forming a loop on either end. Each loop is approximately 1.3 inches in length, 0.9 inches in width, with the right inner edge inclined with the top ridge. Two pieces of adhesive cloth (similar to a standard bandage), approximately one inch long and four inches wide, are needed to cover both of the loops. The pieces of cloth are lined in the center with an absorbent gauze pad for cushioning. After lining the adhesive cloth with the gauze padding, one piece is used to wrap around each wire loop by tucking the bottom under and wrapping the sides around the loop. The final weight of the nose-plug is approximately 2.5 grams, 0.85 ounces. The wearer is able to adjust the nose-plug to the width of his/her nose by squeezing the loops behind their nostrils. This device is new and different from the nose-plugs that are currently available on the market because it is light weight, does not require a strap, fits comfortably behind the nostrils without pinching them, is inconspicuous because it can come in various flesh tones, while actually preventing water from entering the nasal cavities. Previously, nose-plugs have either been made out of hard plastic or hard metal covered in plastic that pinches the nose, which causes great discomfort, bruising, and do not adequately keep water out of the nasal cavities. I swim daily, and ever since I have invented Dol-Phins, I have not suffered from any sinus problems, and other swimmers have even asked me where they can find nose-plugs like mine.

### DRAWING

The drawings that has been filed with the application is a front view and side view of the invention entitled “Dol-Fins”, drawn to show it as is worn on the nose behind the nostrils as is described in the above paragraph. The drawings were created by the inventor, Kathleen Mishler.

I. This device is constructed of light weight wire which can be adjusted to fit comfortably behind the back of the nostrils. This is a unique design because no other nose-plug on the market has this wire loop design that is flexible enough to fit people of almost any size.

II. This device is placed behind the nostrils, which prevents water from entering the nasal cavities. This design is unique because all of the nose-plugs squeeze the nostrils directly in an effort to keep water out.

III. This device can come in many skin tones, which makes the nose-plug more inconspicuous than the nose-plugs that have been marketed to date. Nose-plugs typically come in bright colors, such as white, blue, or yellow, and often come with a head strap. These devices are typically not very aesthetically pleasing.
